(19) United States
(12) Patent Application Publication
(10) Pub. No.: US 2025/0211652 A1
(43) Pub. Date: Jun. 26, 2025

(54) COMPUTER SYSTEM AND METHOD FOR ADVANCING SERVICE PROVISION AND DEVELOPMENT ACROSS A CONTINUUM OF SUBJECT-RELATED SERVICE

(71) Applicant: LizAI Inc., Newton, MA (US)

(72) Inventors: David R. Elmaleh, Newton, MA (US); Duy Khiet Ho, Quincy, MA (US); Trung Tin Nguyen, Saarbrücken (DE)

(73) Assignee: LizAI Inc., Newton, MA (US)

(21) Appl. No.: 18/395,339

(22) Filed: Dec. 22, 2023

(51) Int. Cl. H04L 67/51; G06F 16/332; G06F 16/951

(52) U.S. Cl. CPC H04L 67/51; G06F 16/3329; G06F 16/951

(57) ABSTRACT

A method comprising receiving at least one input message indicative of at least one subject-related keyword, and, in response to receiving the input message, obtaining subject-related information from one or more data repositories, which are hosted by one or more network nodes and contain a plurality of data objects, where each of the plurality of data objects is adapted to be rendered by a network browser. The obtaining comprises accessing at least one prioritized subject-related web service including an integrated search functionality, providing the at least one subject-related keyword to the integrated search functionality, in response to providing the at least one subject-related keyword, a search results page from the at least one prioritized subject-related web service, the search results page including at least one identifier of at least one data object contained in the one or more data repositories.

### BACKGROUND

The provision of healthcare to a patient is normally subject to multiple decisions that are typically taken at different instances, including, for example, through directives of health administration, by members of the medical staff, by developers of pharmaceuticals, or by the patient him-/herself. In this context, providing optimal healthcare to an individual patient is difficult, especially, since for the treatment of a particular disease often various pharmaceutical and/or non-pharmaceutical therapies are available, from which a pre-scribing doctor must choose. Meanwhile, each of these therapies may have varying effects, including varying side effects, on individual patients. Furthermore, many of the effects which a medical treatment may have in the case of an individual patient and their intensity are often unknown or difficult to assess in advance, especially if a patient suffers from multiple diseases each of which demands to be treated simultaneously with the respective other(s).

Furthermore, developers of pharmaceuticals conventionally base their designs on tests performed on predefined test groups. However, the scope of such tests and the size of test groups are practically limited and narrow in comparison to the number of future patients and the range of individual effects which a pharmaceutical may have among them.

In addition, members of health administration, prescribing doctors, developers of pharmaceuticals and patients are often restricted in their decisions to experience, which in many cases has been gained from tests that are performed based on previous expectation of causal interaction.

Analogous problems exist in various non-health-related fields of service and development.

### Summary of the Disclosure

There is thus a need for a technique which facilitates improved subject-related service provision across multiple instances in the continuum of subject-related service.

According to a first aspect, a method, performed by a computer system in a net-work, is provided. The method comprises receiving, by means of one or more network crawler modules of the computer system, at least one input message indicative of at least one subject-related keyword, and, in response to receiving the input message, obtaining, via the network, subject-related information from one or more data repositories, which are hosted by one or more network nodes and contain a plurality of data objects, wherein each of the plurality of data objects is adapted to be rendered by a network browser. Obtaining the subject-related information comprises accessing, via the network, at least one prioritized subject-related web service, the at least one prioritized subject-related web service including an integrated search functionality, providing the at least one subject-related keyword to the integrated search functionality of the at least one prioritized subject-related web service, receiving, in response to providing the at least one subject-related keyword, a search results page from the at least one prioritized subject-related web service, the search results page including at least one identifier of at least one data object contained in the one or more data repositories, crawling, by means of the one or more network crawler modules, the one or more data repositories based on the at least one data object associated with the at least one identifier, wherein the crawling includes rendering, by means of at least one browser engine of the computer system, any data object subject to the crawling, and analyzing, by means of the computer system and using a first machine learning model, a rendered content of each rendered data object to determine subject-related information encoded in each data object, respectively. The method further comprises processing the subject-related information to determine at least one classification indicator associated with the subject-related information, the classification indicator indicative of a classification of the subject-related information with respect to the at least one subject-related keyword using a second machine learning model, and storing the processed subject-related information and the at least one associated classification indicator in a system database, wherein subject-related information stored in the system database is accessible for generating, by means of the computer system and using a third machine learning model, an output message for output towards one or more user terminals.

According to another aspect, a computer program product is provided. The computer program product contains portions of program code which, when executed by a processor of a computer system, configure the computer system to perform the method as presently provided.

According to another aspect, a computer system is provided. The computer system comprises a processor and a data storage device operatively coupled to the processor, the data storage device containing portions of program code which, when executed by the processor, configure the processor to receive, by means of one or more network crawler modules of the computer system, at least one input message indicative of at least one subject-related keyword, and, in response to receiving the input message, obtain, via a network, subject-related information from one or more data repositories, which are hosted by one or more network nodes and contain a plurality of data objects, wherein each of the plurality of data objects is adapted to be rendered by a network browser. Obtaining the subject-related information comprises accessing, via the network, at least one prioritized subject-related web service, the at least one prioritized subject-related web service including an integrated search functionality, providing the at least one subject-related keyword to the integrated search functionality of the at least one prioritized subject-related web service, receiving, in response to providing the at least one subject-related keyword, a search results page from the at least one prioritized subject-related web service, the search results page including at least one identifier of at least one data object contained in the one or more data repositories, crawling, by means of the one or more network crawler modules, the one or more data repositories based on the at least one data object associated with the at least one identifier, wherein the crawling includes rendering, by means of at least one browser engine of the computer system, any data object subject to the crawling, and analyzing, by means of the computer system and using a first machine learning model, a rendered content of each rendered data object to determine subject-related information encoded in each data object, respectively. The portions of program code further configure the processor to process the subject-related information to determine at least one classification indicator associated with the subject-related information, the classification indicator indicative of a relevance of the subject-related information with respect to the at least one subject-related keyword using a second machine learning model, and store the processed subject-related information and the at least one associated classification indicator in a system database, wherein subject-related information stored in the system database is accessible for generating, by means of the computer system and using a third machine learning model, an output message for output towards one or more user terminals.

### BRIEF DESCRIPTION OF THE FIGURES

Further aspects, objectives and advantages of the disclosure become clear from the drawings and the detailed description. There is shown in:

FIG. 1 a computer system in a networked computing environment, according to an example;

FIG. 2 a method, according to an example; and

FIGS. 3 and 4 computer systems in networked computing environments, according to further examples.

### DETAILED DESCRIPTION

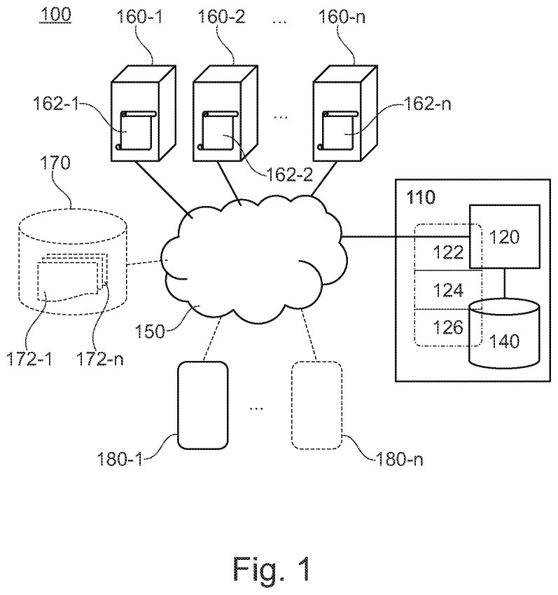

FIG. 1 shows schematically and exemplarily a networked computing environment 100. The networked computing environment 100 comprises a computer system 110, which is communicatively coupled to a network 150, such as the Internet. Further connected to the network 150 is a plurality of network nodes, which include multiple servers 160-1, 160-2, 160-n. Each of the servers stores at least one data object 162-1, 162-2, 162-n, which pertain to one or more data repositories hosted by the servers 160-1, 160-2, 160-n. In some examples, at least some of the data objects 162-1, 162-2, 161-n pertain to a same data repository, such as a database which is managed by a third party and which is hosted in a distributed manner by various of the servers 160-1, 160-2, 160-n. Moreover, in some examples, each of at least some of the data objects 162-1, 162-2, 162-n belongs to a different data repository. The data objects 162-1, 162-2, 162-n are accessible via the network 150, for example, by means of a network browser of a computer system connected to the network 150, such as the computer system 110. In some examples, the data objects 162-1, 162-2, 162-n include one or more websites, which can be accessed and rendered by means of a network browser, such as public Internet websites, Internet websites with privileged access, etc.

The networked computing environment 100 further includes a user terminal 180-1 which is communicatively connected to the network 150. The user terminal 180-1 may include any type of user device which is suited for communicating data via the network 150, outputting received data towards a user via a user interface of the user terminal 180-1 and receiving user input data from the user via the user interface and transmitting received user input data via the network 150. Those types include mobile computing devices, such as smartphones, notebooks and/or tablet computers, stationary user terminals, such as computer stations with a user interface, etc.

As indicated by the dashed lines in FIG. 1, the networked computing environment 100 may in some examples optionally include additional user terminals 180-n. Each of the additional user terminals 180-n is communicatively connected to the network 150.

Moreover, as indicated by the dashed lines in FIG. 1, the networked computing environment 100 may in some examples optionally include at least one subject-related, non-public database 170. The non-public database 170 stores non-public information which is encoded in one or more non-public data objects 172-1, 172-n. In an example, the non-public database 170 is a clinical database storing information about one or more patients, such as patient-specific bio-parametric data, patient-specific health data, patient-specific medical treatment data, non-patient-specific health data, non-patient-specific medical treatment data, etc., which are encoded in non-public data objects 172-1, 172-n.

The computer system 100 includes a processor 120 and a data storage device 140 operatively connected to the processor 120. The data storage device 140 stores program code which is executable by means of the processor 120. When the processor 120 executes the program code, the processor 120 performs operations which functionally constitute a network crawler module 122, a browser engine 124 and the first machine learning model 126, as described in more detail below.

When in operation, the computer system 110 is configured to receive an input message which is indicative of at least one subject-related keyword by means of the network crawler module 122. In an example, the input message has been generated by means of the user terminal 180-1 and transmitted to the computer system 110. For example, the input message has been generated based on a user query which a user of the user terminal 180-1 provided via the user interface of the user terminal 180-1 and which contains the subject-related keyword. In other examples, the keyword has been generated by means of a keyword generator module (not shown) of the computer system 110 and is provided by way of an input message to the network crawler module 122.

Upon receiving the input message, the computer system 110 retrieves subject-related information from any one of the data repositories connected to the network 150. For this purpose, the network crawler module 122 accesses via the network 150 a prioritized subject-related web service which includes an integrated search functionality. The prioritized subject-related web service is in some examples determined by the computer system 110 based on the subject-related keyword indicated by the input message. In other examples, including examples in which the computer system 110 is dedicated to an operation with respect to a particular subject, the prioritized subject-related web service has been predetermined and stored, for example, in the data storage device 140.

A priority of the prioritized subject-related web service is selected in some examples in accordance with a significance, including, for example, an amount, a diversity, a proportion, an actuality, an authority and/or an associated trustworthiness, of data objects managed by the prioritized subject-related web service with respect to a subject to which the subject-related keyword relates. In examples in which the subject-related keyword is a health-related keyword and/or the computer system 110 is dedicated to operation in connection with health-related information, the prioritized subject-related web service includes any one of a public web service provided by a governmental or supranational health organization, a digital catalog of one or more medical libraries, etc.

The computer system 110 provides the keyword to the integrated search functionality of the prioritized subject-related web service. Providing the keyword is performed, for example, by the network crawler module 122 or the browser engine 124. In response to providing the keyword, the computer system 110 receives a search results page from the prioritized subject-related web service via the network 150. Depending on the outcome of the search performed by the integrated search functionality of the prioritized subject-related web service, the search results page typically includes a plurality of identifiers, such as network links or URLs, associated with some of the data objects 162-1-162-n in the data repositories hosted by servers 160-1-160-n.

The network crawler module 122 performs crawling in data repositories which are hosted by the servers 160-1-160-n. Crawling is performed in such manner that the network crawler module 122 initially selects one of the identifiers included in the search results page and accesses the a corresponding data object 162-1-162-n. Crawling is then performed on that data object in such way that the data object is rendered by means of the browser engine 124 and the rendered content of the data object is analyzed. References, such as network links, URLs, etc., in the rendered content pointing to further data objects contained in a data repository are then identified and utilized to subsequently access thus referenced data objects and continue crawling in an analogous manner on the referenced data objects. When a data object is reached that does not contain any reference to further data objects, one or more steps backwards are made in the referencing tree until an identifier is determined to a data object which has not yet been subject to crawling, potentially back until the search results page, where another identifier is then chosen, and so on.

In parallel or successively to the crawling, the rendered content of each data object is analyzed to determine subject related information encoded in each respective data object. Determining the subject related information in the rendered content is performed using the first machine learning model 126, which has been trained for this purpose.

Subject-related information which has been determined in the rendered content of the data object is processed by means of the computer system 110 for determining one or more classification indicators associated with a respective subject-related information. The classification indicators are indicative of a classification of a respective subject-related information with respect to the subject-related keyword. Classifying the determined subject-related information is performed using a second machine learning model which is stored, for example, in the data storage device 140 together with the first machine learning model.

The processed, in particular classified, subject-related information is subsequently stored together with the one or more associated classification indicators in the system database comprised by the storage device 140. Processed subject-related information which has been stored in the system database 140 is accessible to the computer system 110 for further use. This includes a use in generating an output message by the computer system 110 for output towards the user terminal 180-1 and/or any other user terminal connected to the network 150 presently or at a later moment. The output message is generated using a third machine learning model stored in the data storage device 140.

In some examples the classification indicators associated with a processed subject-related information is indicative of a determined relevance of the subject-related information to the subject-related keyword based on which the crawling has been performed. The relevance is determined using the second machine learning model, for example. Generating an output message for output towards the user terminal 180-1 in response to the user query using the third machine learning model is performed in some examples in accordance with a relevance associated with each of various items of processed subject-related information stored in the data storage device 140.

In some examples, crawling the one or more data repositories in response to receiving an input message is performed until a stop condition is fulfilled. The stop condition includes in some examples an expiry of a pre-set crawling period which is associated with the input message. For example, the data storage device 140 can contain a mapping between types of input messages and one or more different crawling periods. In other examples, the input message further includes a crawling period indicator indicative of the crawling period associated with the input message.

Applying a short crawling period is advantageous for obtaining processed subject-related information promptly. In combination with the use of one or more prioritized subject-related web services for initiating the crawling, significance of the crawled data objects to the subject-related keyword is enhanced. Conversely, a long crawling period allows for a greater amount of data objects to be crawled, thus providing for a larger amount of processed subject-related information, which, for example, enables the generation of more significant output messages.

Rendered content of the data object typically includes textual content. However, in many cases, the data object also includes non-textual content, such as image data, video data and/or audio data, which may be rendered by the browser engine 124. When analyzing the rendered content, the computer system 110 is adapted to determine subject-related information encoded in any of textual, video and or audio format. In addition, in some examples, the computer system 110 is adapted to generate a textual representation of non-textual content included in a data object and determine subject-related information which is encoded in the textual representation.

By rendering and analyzing non-textual content in addition to textual content of a data object, information encoded in the data object is processed more comprehensively. As such, data objects that have been selected based on the prioritized subject-related web service, which can be assumed to contain information of prioritized relevance, are analyzed to an increased extent. Especially in a limited crawling interval, this contributes to a relevance of the information which is obtained and processed by the computer system 110 within a given crawling interval.

As indicated by the dashed lines in FIG. 1, the computer system 110 may be accessed by a plurality of users associated with a plurality of user terminals 180-1-180-n. In the case that multiple users transmit input messages towards the computer system 110, a plurality of crawling requests may coincide. For managing coinciding crawling requests, the computer system 110 includes in some examples a queuing facility for queuing a plurality of incoming input messages and processing queued input messages subsequently, for example, after expiry of each corresponding crawling interval.

In some examples, the computer system 110 employs a fourth machine learning model. The fourth machine learning model is adapted to determine the subject-related keyword from complex user input provided to the user terminal 180-1, for example, a voice input or complex textual input. The fourth machine learning model may be stored in the data storage device 140. In other examples, the fourth machine learning model may reside on the user terminal 180-1, for example, after transmission of machine learning data for installation on the user terminal 180-1.

When the computer system 110 is in operation, each of the first, second, third and/or fourth machine learning model can be trained in some examples individually, pairwise or in larger groups in an interacting manner. For example, an improved processing of determined subject related information with respect to a relevance regarding a specific subject-related keyword can be used in determining a keyword from complex user input, for example, in consideration of further context data, and the like.

In examples in which the networked computing environment 100 comprises the nonpublic database 170, the computer system 110 accesses nonpublic data objects 172-1-172-n during a triggered crawling and/or in idle times, i.e., when currently no input message is to be processed. The computer system 110 analyzes in that case the data objects 172-1-172-n with respect to information related to a current or previous keyword and processes such information for storage in the data storage device 140. The nonpublic information is and that case available, subject to privacy protection, for training any of the first, second, third and/or fourth machine learning models and/or for generating an output message, and for other uses, as described below.

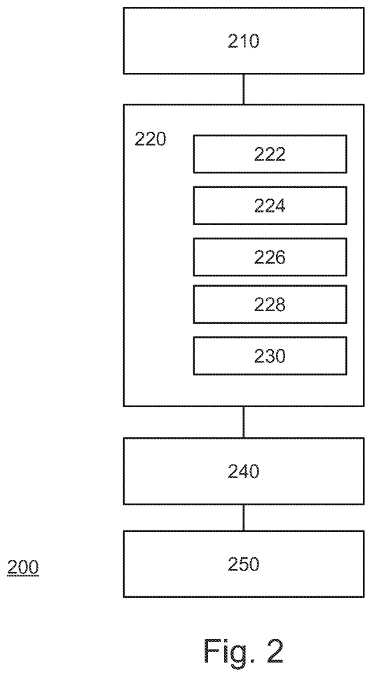

FIG. 2 shows a flow diagram of a method 200 performed by computer system in a network, such as computer system 110 in the networked computing environment 100.

The method 200 includes receiving at least one input message indicative of at least one subject-related keyword by means of one or more network crawling modules of the computer system, step 210. The method 200 further includes obtaining, in response to receiving the input message and via the network, subject-related information from one or more data repositories, which are hosted by one or more network nodes and contain a plurality of data objects, step 220. Each of the plurality of data objects is adapted to be rendered by a network browser, as described above in connection with FIG. 1.

Obtaining subject-related information, step 220, comprises a plurality of operations, including accessing, via the network, at least one prioritized subject-related web service which includes an integrated search functionality, step 222. Obtaining the subject-related information, step 220, further comprises providing the at least one subject-related keyword to the integrated search functionality of the prioritized subject-related web service, step 224, and receiving the search results page from the at least one prioritized subject-related web service in response to providing the at least one subject-related keyword, step 226. The search results page includes at least one identifier of at least one data object contained in the one or more data repositories.

Obtaining the subject-related information, step 220, further comprises crawling the one or more data repositories by means of the one or more network crawling modules based on the at least one data object associated with the at least one identifier in the search results page, step 228. The calling includes rendering any data object which is subject to the crawling by means of at least one browser engine of the computer system.

Obtaining the subject-related information, step 220, further comprises analyzing the rendered content of each rendered data object by means of the computer system and using a first machine learning model to determine subject-related information encoded in each data object, step 230.

The method 200 further comprises processing the subject-related information to determine at least one classification indicator which is associated with the subject-related information, step 240. The classification indicator is indicative of a classification of the subject-related information with respect to the at least one subject-related keyword using a second machine learning model.

The method 200 further comprises storing the processed subject-related information and the at least one associated classification indicator in the system database, step 250. The subject-related information, when stored in the system database, is accessible for generating an output message for output towards one or more user terminals by means of the computer system and using a third machine learning model.

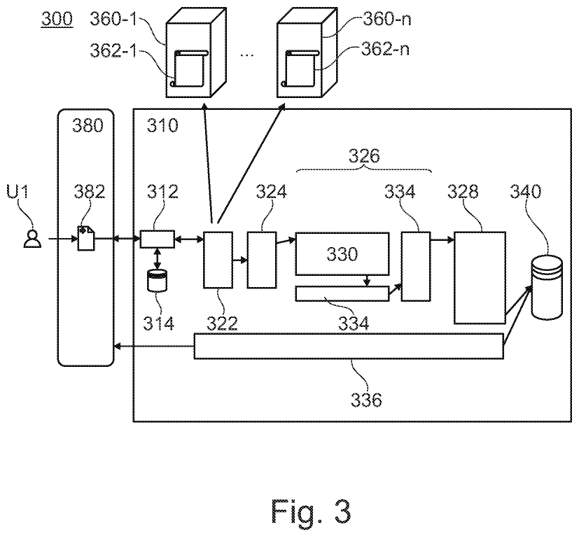

FIG. 3 shows schematically and exemplarily a networked computing environment 300 according to another example. The networked computing environment 300 comprises a computer system 310 which is communicatively connected via a network (not shown) to a plurality of servers 360-1-360-n storing a plurality of data objects 362-1-362-n. Moreover, the computer system 310 is communicatively connected by the network to a user terminal 380 associated with a user U1. Concerning the aforesaid components of the networked computing environment 300, the preceding description of the networked computing environment 100, the computing system 110, servers 160-1 to 160-n and the user terminal 180-1 shown in FIG. 1 apply correspondingly unless otherwise clear from the figures and the following description.

The user terminal 380 includes a user interface and communication logic 382. The user interface and communication logic 382 is adapted to receive user input from the user U1 and output information towards the user U1. It is further adapted to transmit and receive data via one or more communication networks. In the shown example, the user terminal 380 receives user input from user U1 which is indicative of a keyword, for example, a subject-related such query. The user terminal 380 transmits an input message indicative of the keyword towards the computer system 310.

The computer system 310 comprises a queuing module 312 which is connected to a scheduler 314. The input message transmitted by the user terminal 380 is received by the queuing module 312 which queues the input message with other incoming input messages from the same user terminal 380 or other user terminals. For example, incoming input messages may be queued by the queuing module 312 in a first-in-first-out manner. Furthermore, the scheduler 314 is adapted in some examples to manage queued input messages by controlling a time interval during which each input message is processed by the computer system 310 before the processing changes to a next input message in the queue. In times when no input message is received or no received input message is being processed, the scheduler 314 determines in some examples an automatic generation of a keyword by the computer system 310, for example, for enhancing a system database during idle times of the computer system 310.

An input message which is to be processed is forwarded from the queuing module 312 towards the crawler module 322. Network links which are identified by the crawler module 322 in data objects 362-1 to 362-n when crawling data repositories hosted by the servers 360-1 to 360-n are transmitted to the browser engine 324 of the computer system 310. The browser engine 324 renders the content associated with received network links, such as URLs, etc.

The rendered content is analyzed with respect to subject-related information using a first machine learning model 326 of the computer system 310. Analyzing the rendered content includes detection of textual, graphical and/or audio information included in the rendered content using a detector module 330 of the computer system 310. The detected information is then subjected to data extraction by means of a data extractor 334 of the computer system 310. The extracted data is forwarded to a text inverter 334 of the computer system 310, which converts textual, graphical and/or audio information detected in the rendered content and extracted by the data extractor 334 into a unified textual format for further processing.

The subject-related information extracted from the rendered content and converted into a unified textual format is subsequently processed to determine one or more classification indicators associated with a respective item of subject-related information. This is performed using a second machine learning model 328 of the computer system 310. A classification indicator includes in some examples a determined relevance of the respective item of subject-related information with respect to the keyword indicated by the input message.

The processed, in particular classified, subject related information is subsequently stored in a system database 340 of the computer system 310 together with the associated classification indicators. Information stored in the system database 340 is accessible to a third machine learning model 336 of the computer system 310. The third machine learning model 336 has been trained to generate an output message for output towards user terminal 380, for example, in a response message to the user query input to mobile terminal 380, based at least partially on the keyword.

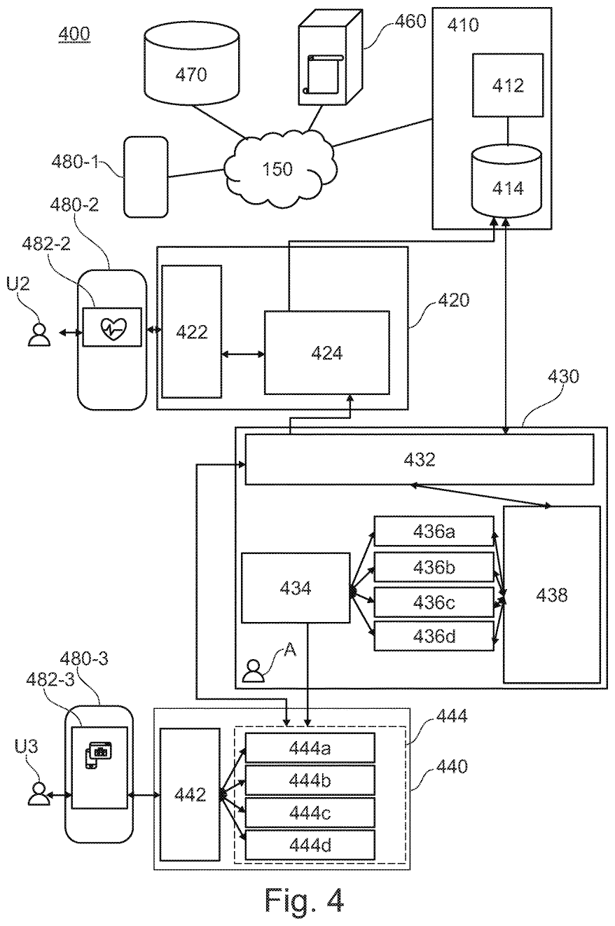

FIG. 4 shows schematically and exemplarily a networked computing environment 400 according to another example. The networked computing environment 400 comprises a network 150 to which a computer system 410, one or more servers 460 storing a plurality of data objects, a nonpublic database 470, and at least one first user terminal 480-1 are connected. Regarding these components of the networked computing environment 400, the previous description of corresponding networked components in connection with FIGS. 1 to 3 applies correspondingly unless otherwise clear from the drawings and the following description.

The networked computing environment 400 further comprises a real-time monitoring and suggestion platform 420. The real-time monitoring and suggestion platform 420 is connected to the computer system 410 via the network 150. Moreover, the real-time monitoring and suggestion platform 420 has operational access to a system database 414 of the computer system 410. In some examples, as shown in FIG. 4, the real-time monitoring and suggestion platform 420 is implemented separate from the computer system 410, for example, hosted by a separate network node connected to the network 150. In other examples, the real-time monitoring platform 420 is at least partially implemented in the computer system 410.

The real-time monitoring and suggestion platform 420 is communicatively accessible and/or has communicative access to one or more second user terminals 480-2 associated with one or more second users U2. The user terminal 480-2 includes a real-time monitoring functionality 482-2 which is configured to monitor in real-time at least one health-related parameter of the user U2, such as a pulse rate, an activity, a body temperature, a blood composition, or the like. Furthermore, the user terminal 480-2 is configured to transmit associated monitoring data to the real-time monitoring and suggestion platform 420.

The real-time monitoring and suggestion platform 420 comprises a monitoring data converter 422 which receives the real-time monitoring data transmitted by the user terminal 480-2 and which converts received monitoring data into health-related information in a format that is processable by a fifth machine learning model 424 of the realtime monitoring and suggestion platform 420. The fifth machine learning model 424 has been trained to analyze the health-related information based on the monitoring data with respect to a user-specific health condition of the user U2. In some examples, the fifth machine learning model 424 has further been trained to analyze monitoring data received from a plurality of user terminals 480-2 with respect to the user-specific health condition of an associated plurality of users U2 and to determine non-user-specific processed health information, such as classified health information relating to a group of monitored health parameters.

The real-time monitoring and suggestion platform 420 communicates the processed health-related information to the computer system 410 for storage of the healthrelated information in the system database 414. Health-related information which is received by the computer system 410 from the real-time monitoring and suggestion platform 420 and which is stored in the system database 414 is accessible for any functionality of the computer system 410, such as for training remaining machine learning models of the computer system 410, based on real-time monitoring data of users U2.

The real-time monitoring and suggestion platform 420 is further configured to generate, using the fifth machine learning model 424, health-related suggestions for transmission to the user terminal 480-2 to be output towards the user U2 corresponding to received monitoring data. Health-related suggestions include, for example, suggestions regarding a health-related behavior of the user U2, including physical activity, diet, therapeutical and/or medical treatment, for example. To facilitate improved suggestions, the computer system 410 and the real-time monitoring and suggestion platform 420 are adapted to enable continued training of the fifth machine learning model 424 using any types of processed health-related information stored in the system database 414.

In some examples different from the above examples, the real-time monitoring and suggestion platform 420 is implemented at least partially on the one or more user terminals 480-2.

The networked computing environment 400 further includes an analysis and development platform 430. As shown in FIG. 4, the analysis and development platform 430 is in some examples implemented separate from the computer system 410 and is communicatively coupled to the computer system 410 via the network 150. In other examples, at least parts of the analysis and development platform 430 are implemented in the computer system 410.

The analysis and development platform 430 provides an interface for an administrator A to determine and/or define criteria for classifying health-related information available in the networked computing environment 400, for example, with respect to a processing of the available health-related information in terms of any of the purposes described herein and/or other purposes, including providing output messages to users, extracting analysis data, planning treatment, drug development, and the like.

In particular, the analysis and development platform 430 comprises a task manager module 432, a sixth machine learning model 434, a plurality of adjustable development criteria settings 436a to 436d, and one or more machine learning applications 438.

As shown in FIG. 4, the development criteria settings 436a to 436d include, for example, criteria such as one or more underlying causes of disease 436a, one or more mechanisms of actions of at least one medicine 436b, increased potency of one or more medicines 436c, and precision doses of one or more treatments to reduce side effects 436d. The at least one machine learning application 438 acts between the drug development criteria 436 and the task manager module 432. Moreover, the sixth machine learning model 434 is adapted to act between the development criteria settings and a healthcare and drug development platform 440 of the networked computing environment 400 as described below. Furthermore, the task manager module 432 communicates with the fifth machine learning model 424 of the real-time monitoring and suggestion platform 420, for example, in accordance with adjustments performed by the administrator A of the analysis and development platform 430.

The networked computing environment 400 further comprises a healthcare and drug development platform 440. In the shown example, the healthcare and drug development platform 440 is connected to the computer system 410 via the analysis and development platform 430 to which the health and development platform 440 is communicatively connected. This implementation is chosen for convenience. However, in other implementations the healthcare and development platform 440 is connected directly to the computer system 410 via the network 150 while using functionalities of the analysis and development platform 440 via the computer system 410.

The healthcare and development platform 440 is accessible to one or more user terminals 480-3 associated with a user U3. The healthcare and development platform 440 is configured to receive requirements specifications from the user terminal 480-3 which have been input by the user U3, for example, with respect to a pharmaceutical and/or medical treatment. Requirements specification received from the user terminal 480-3 are input to a sixth machine learning model 442 of the healthcare and development platform 440. The sixth machine learning model 442 analyzes the received requirements specification with respect to requirements criteria 444a to 444d and transmits corresponding processed health-related information in accordance with one or more identified requirements criteria to the task manager module 432 of the analysis and development platform 430. Requirements criteria 444 include, for example, suggestions for a precision treatment service 444a, prediction of clinical outcomes 4404b, targeted drug development 444c and/or accelerated clinical studies 444d. Processed health-related information with respect to requirements criteria 444 which has been received by the task manager module 432 is being processed by the computer system 410 using information included in the system database 414 for providing an output message to the healthcare and drug development platform 440, which is to be output to the user U3 in response to the provided requirements specifications.

By means of a networked computing environment as described above, in particular by use of a computer system as described above, subject-related information, in particular health-related information, which is obtained at various stages, both by detection and monitoring of actual conditions, reactions, clinical studies, and the like, and by receiving related requirements and/or query data, across the continuum of subject-related services, is being acquired and jointly processed, using multiple trained machine learning models adapted to various instances in that continuum. In this way, the proposed networked computing environment facilitates improved data processing and data provision to any of multiple instances and/or users in the continuum of subject-related service. While the above examples have been described in connection with health-related services, it will be understood that the described techniques can be advantageously applied to any other subject, while the same or analogous advantages are achievable.

We claim:

1. A method, performed by a computer system in a network, the method comprising:
receiving, by means of one or more network crawler modules of the computer system, at least one input message indicative of at least one subject-related keyword;
in response to receiving the input message, obtaining, via the network, subject-related information from one or more data repositories, which are hosted by one or more network nodes and contain a plurality of data objects, wherein each of the plurality of data objects is adapted to be rendered by a network browser and wherein obtaining the subject-related information comprises:
accessing, via the network, at least one prioritized subject-related web service, the at least one prioritized subject-related web service including an integrated search functionality,
providing the at least one subject-related keyword to the integrated search functionality of the at least one prioritized subject-related web service,
receiving, in response to providing the at least one subject-related keyword, a search results page from the at least one prioritized subject-related web service, the search results page including at least one identifier of at least one data object contained in the one or more data repositories,
crawling, by means of the one or more network crawler modules, the one or more data repositories based on the at least one data object associated with the at least one identifier, wherein the crawling includes rendering, by means of at least one browser engine of the computer system, any data object subject to the crawling, and analyzing, by means of the computer system and using a first machine learning model, a rendered content of each rendered data object to determine subject-related information encoded in each data object, respectively,
processing the subject-related information to determine at least one classification indicator associated with the subject-related information, the classification indicator indicative of a classification of the subject-related information with respect to the at least one subject-related keyword using a second machine learning model, and
storing the processed subject-related information and the at least one associated classification indicator in a system database, wherein subject-related information stored in the system database is accessible for generating, by means of the computer system and using a third machine learning model, an output message for output towards one or more user terminals.

2. The method of claim 1, wherein:
the at least one input message is received from a user terminal via the network, and/or
the at least one keyword has been generated by means of a keyword generator module of the computer system, and the at least one input message is received from the keyword generator module.

3. The method of claim 1, wherein the at least one subject-related keyword, the subject-related information and the at least one subject-related prioritized web service are related to a same subject, wherein the same subject includes a health-related subject.

4. The method of claim 1, wherein the classification indicator is indicative of a determined relevance of the subject-related information to the at least one subject-related keyword, the relevance determined using the second machine learning model.

5. The method of claim 1, wherein the crawling is performed until a fulfilment of a stop condition, in particular an expiry of a pre-set crawling period associated with the input message, is determined.

6. The method of claim 1, wherein accessing the at least one prioritized subject-related web service includes accessing, in particular successively, a plurality of prioritized subject-related web services, each of the prioritized subject-related web services including an integrated search functionality, and providing the at least one subject-related keyword comprises providing the at least one subject-related keyword to the integrated search functionality of each of the prioritized subject-related web services.

7. The method of claim 1, further comprising training, by means of the computer system, at least one of the first machine learning model, the second machine learning model or the third machine learning model using the subject-related information and associated classification indicators stored in the system database.

8. The method of claim 1, wherein the rendered content of at least one rendered data object includes non-textual content and wherein analyzing the rendered content includes generating a textual representation of at least parts of the non-textual content and determining subject-related information encoded in the textual representation.

9. The method of claim 1, wherein the at least one prioritized subject-related web service contains one or more web services provided by a governmental health organization.

10. The method of claim 1, wherein crawling the one or more data repositories based on the at least one data object associated with the at least one identifier comprises crawling the one or more data repositories starting from the at least one data object associated with the at least one identifier and continuing according to at least one URL determined based on the at least one data object, the at least one URL associated with at least one other data object.

11. The method of claim 1, wherein receiving the at least one input message includes receiving a plurality of input messages from one or more user terminals, and wherein the method further comprises queuing the plurality of input messages for processing the plurality of input messages successively.

12. The method of claim 1, wherein the network comprises the Internet.

13. The method of claim 1, wherein the plurality of network nodes comprises one or more servers, and the at least one data repository comprises a repository of websites, wherein the at least one data object is comprised by a website stored by means of the one or more servers.

14. The method of claim 1, wherein the plurality of network nodes further comprises at least one clinical database which stores one or more clinical data objects, wherein the method further comprises retrieving at least one of the one or more clinical data objects from the at least one clinical database and storing, as subject-related information, health-related information determined based on the at least one clinical data object in the system database.

15. The method of claim 1, further comprising:
receiving from at least one user terminal user monitoring data indicative of at least one health-related condition of a user detected by means of the user terminal, and
storing, as subject-related information, health-related information determined based on the user monitoring data in the system database.

16. The method of claim 1, wherein the keyword is included in a health-related query contained in the input message and wherein the method further comprises generating a response message based on the health-related information stored as subject-related information in the system database using the third machine learning model for output towards the user terminal.

17. The method of claim 16, wherein the health-related query relates to an individual condition of a user of the user terminal or of another person and the response message is indicative of a personalized treatment suggestion for the user or the other person.

18. The method of claim 16, wherein the health-related query relates to one or more constraints in a medical treatment development and the response message is indicative of a suggested medical treatment scheme, in particular including a suggested pharmaceutical composition.

19. The method of claim 1, wherein the input message comprises voice input data, and wherein the method further comprises processing the voice input data using a fourth machine learning model to determine a keyword based on the voice input data.

20. The method of claim 4, wherein training any of the machine learning models is performed at least partially with respect to one or more of the following criteria:
one or more underlying causes of a disease,
one or more mechanisms of actions of one or more medicines,
an increased potency of one or more medicines,
a precision dose of one or more treatments to reduce side effects.

21. The method of claim 1, wherein the computer system comprises a distributed computing architecture and wherein at least some of the functionalities of the computer system are implemented at different instances of the distributed computing architecture.

22. A computer program product containing portions of program code which, when executed by a processor of a computer system, configure the computer system to perform the method of claim 1.

23. A computer system comprising a processor and a data storage device operatively coupled to the processor, the data storage device containing portions of program code which, when executed by the processor, configure the processor to perform the following steps:
receiving, by means of one or more network crawler modules of the computer system, at least one input message indicative of at least one subject-related keyword;
in response to receiving the input message, obtaining, via a network, subject-related information from one or more data repositories, which are hosted by one or more network nodes and contain a plurality of data objects, wherein each of the plurality of data objects is adapted to be rendered by a network browser and wherein obtaining the subject-related information comprises:
accessing, via the network, at least one prioritized subject-related web service, the at least one prioritized subject-related web service including an integrated search functionality,
providing the at least one subject-related keyword to the integrated search functionality of the at least one prioritized subject-related web service,
receiving, in response to providing the at least one subject-related keyword, a search results page from the at least one prioritized subject-related web service, the search results page including at least one identifier of at least one data object contained in the one or more data repositories,
crawling, by means of the one or more network crawler modules, the one or more data repositories based on the at least one data object associated with the at least one identifier, wherein the crawling includes rendering, by means of at least one browser engine of the computer system, any data object subject to the crawling, and
analyzing, by means of the computer system and using a first machine learning model, a rendered content of each rendered data object to determine subject-related information encoded in each data object, respectively,
processing the subject-related information to determine at least one classification indicator associated with the subject-related information, the classification indicator indicative of a relevance of the subject-related information with respect to the at least one subject-related keyword using a second machine learning model,
storing the processed subject-related information and the at least one associated classification indicator in a system database, wherein subject-related information stored in the system database is accessible for generating, by means of the computer system and using a third machine learning model, an output message for output towards one or more user terminals.
